(19) United States
(12) Patent Application Publication
(10) Pub. No.: US 2016/0143914 A1
(43) Pub. Date: May 26, 2016

(54) Nanoparticles for Encapsulation and Delivery of Bioactive Compounds and Compositions Thereof

(71) Applicant: EMORY UNIVERSITY, Atlanta, GA (US)

(72) Inventors: Yiqing WANG, Duluth, GA (US); Shuming NIE, Atlanta, GA (US)

(73) Assignee: Emory University, Atlanta, GA (US)

(21) Appl. No.: 14/899,359

(22) PCT Filed: Jun. 12, 2014
(86) PCT No.: PCT/US2014/042225

§ 371 Date: Dec. 17, 2015

Related U.S. Application Data
(60) Provisional application No. 61/834,449, filed on Jun. 13, 2013.

(51) Int. Cl. A61K 31/537; A61K 47/10; A61K 47/36; A61K 45/06; A61K 9/51

(52) U.S. Cl. CPC A61K 31/537; A61K 45/06; A61K 9/5146; A61K 9/5161; A61K 47/36; A61K 47/10

(57) ABSTRACT

In certain embodiments, this disclosure relates to nanoparticles for drug-delivery of cytotoxic anti-cancer compounds. In certain embodiments, the nanoparticle comprises a maytansinoid and an acetalated polysaccharide-polyethylene glycol conjugate. This disclosure also relates to methods for treatment of infection and cancer are also claimed, wherein a cytotoxic compound is en capsulated in a nanoparticle is degraded at the intended target, but otherwise stable, releasing the cytotoxic compound. Compositions comprising the nanoparticles and cytotoxic drugs are also considered.

### CROSS REFERENCE TO RELATED APPLICATIONS

This application Claims priority to U.S. Provisional Application No. 61/834,449, filed 13 Jun. 2013 hereby incorporated by reference in its entirety.

### ACKNOWLEDGEMENT

This invention was made with government support under Grant No. R01 CA163256 awarded by the National Institutes of Health. The government has certain rights in the invention.

### BACKGROUND

Maytansinoids are a class of compounds which possess a distinct cytotoxicity to bacterial infections and cancer cells and carry potential for the treatment of cancers and bacterial infections. However, the specificity and biodistribution of these compounds has been insufficient to allow for practical use. Recent efforts to develop drug delivery methods which would allow maytansinoids and other cytotoxic compounds to be effective cancer treatments have been investigated. Adolf et al. report compositions and methods for treating cancer using maytansinoid immunoconjugates (WO 2004/018000).

The use of polymers to form nanoparticles has also been explored as a drug delivery technique for cytotoxic compounds. These methods have either encapsulated cytotoxic materials within the nanoparticle, or alternatively attached the cytotoxic compounds to the exterior nanoparticle surface. Nanoparticles which are pH-sensitive offer a mechanism for releasing the cytotoxic compounds at the target site in order to illicit an improved biodistribution and specificity.

Duong et al. report functionalizing biodegradable dextran scaffolds using living radical polymerization as nanoparticles for the delivery of therapeutic molecules. Mol Pharm, 2012, 9(11):3046-6.

Broaders et al. report acetalated dextran is a chemically and biologically tunable material for particulate immunotherapy. PNAS, 2009, 106(14):5497-5502.

Frechet et al. report acid-degradable and bioerodible modified polyhydroxylated materials (US 2011/0229550). See also, WO 2010/005847, EP 1871423, US 2013/0058960, US 2012/0302516, WO 2005/121196, U.S. Pat. No. 8,263,664, U.S. Pat. No. 7,368,565.

References cited herein are not an admission of prior art.

### SUMMARY

In certain embodiments, this disclosure relates to nanoparticles for drug-delivery of cytotoxic anti-cancer compounds. In certain embodiments, the nanoparticle comprises a maytansinoid and an acetalated polysaccharide-polyethylene glycol. This disclosure also relates to methods for treatment of infection and cancer are also claimed, wherein a cytotoxic compound is encapsulated in a nanoparticle that is degraded at the intended target, but otherwise stable, releasing the cytotoxic compound. Compositions comprising the nanoparticles and cytotoxic drugs are also considered.

In certain embodiments, this disclosure considers a particle comprising a maytansinoid and an acetalated polysaccharide-polyethylene glycol.

In certain embodiments, the disclosure also relates to such a particle wherein the acetalated polysaccharide-polyethylene glycol conjugate comprises monomers of Formula I:

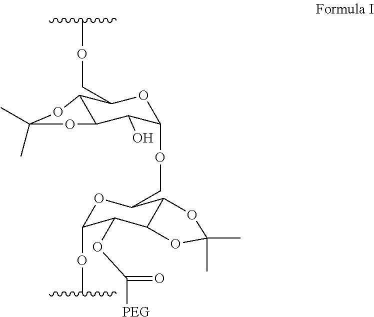

In certain embodiments, this disclosure also considers particles with a size of around 40-400 nanometers.

Certain embodiments consider a particle wherein the acetalated polysaccharide-polyethylene glycol comprises polyethylene glycol fragments with a molecular number of about 4-6 kilodaltons.

In certain embodiments, this disclosure relates to the nanoparticle wherein the acetalated polysaccharide-polyethylene glycol comprises of a dextran polymer with a molecular number of about 5-7 kilodaltons.

This disclosure also considers a nanoparticle wherein the acetalated polysaccharide-polyethylene glycol is stable at physiological pH and unstable in mildly acidic conditions. Some instances of this disclosure consider the nanoparticle wherein the maytansinoid is selected from maytansine, ansamitocin, mertansine, salts, or derivatives optimally substituted with one or more substituents.

In certain embodiments, this disclosure considers the nanoparticle wherein the maytansinoid is a compound of Formula II:

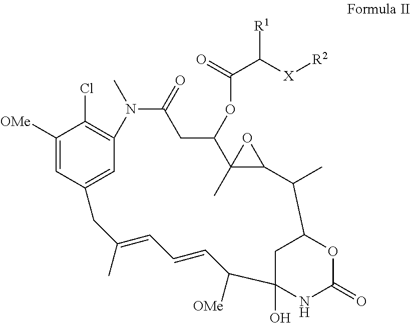

or salts thereof; wherein

R¹ and R² are each independently selected from hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, heterocyclyl, polypeptide, or antibody, wherein R¹ and R² are optionally substituted with one or more of the same or different, J; and wherein

J is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl; and wherein

X is any of oxygen, sulfur, or nitrogen; optionally substituted with one or more of the same or different J.

This disclosure also independently contemplates the nanoparticle comprising a maytansinoid of Formula II wherein X is nitrogen, wherein X is nitrogen substituted with a methyl group, wherein R² is carboxymethyl, and wherein R¹ is methyl.

In certain embodiments, this disclosure contemplates a pharmaceutical composition comprising a nanoparticle, such as those described. Pharmaceutical compositions further comprising an anti-cancer agent are also considered.

In certain embodiments, this disclosure also considers a method of treating cancer or inhibiting a proliferative growth comprising administering to a subject in need of such treatment an effective amount of a pharmaceutical composition. This disclosure also contemplates the method wherein the cancer is selected from of bladder cancer, breast cancer, colon cancer, endometrial cancer, leukemia, lung cancer, melanoma, non-Hodgkin lymphoma, pancreatic cancer, prostate cancer, rectal cancer, renal cell cancer, and thyroid cancer.

In certain embodiments, this disclosure relates to methods herein administering pharmaceutical products herein is done in combination with one or more anticancer agents.

In certain embodiments, this disclosure relates to the production of a medicament for use in treating cancer.

### BRIEF DESCRIPTION OF THE FIGURES

FIG. 1 shows data on experiments. Tumor size of mice treated with a) the dosage of 0.5 mg/kg; and b) 0.75 mg/kg; photographs of mice treated with c) 0.5 mg/kg and d) 0.75 mg/kg. (Dosage calculated based on DM4Me equivalence.)

FIG. 2 shows a scheme for the synthesis of acetalated Dextran-PEG copolymers.

FIG. 3 shows a ¹H NMR of PEG-Dextran in d-DMSO.

FIG. 4 shows ¹H NMR of PEG-Acetylated-Dextran (ADP) in d-DMSO.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

Prior to describing the various embodiments, the following definitions are provided and should be used unless otherwise indicated.

As used herein, “alkyl” means a noncyclic straight chain or branched, unsaturated or saturated hydrocarbon such as those containing from 1 to 22 carbon atoms, while the term “lower alkyl” or “C₁₋₄alkyl” has the same meaning as alkyl but contains from 1 to 4 carbon atoms. The term “higher alkyl” has the same meaning as alkyl but contains from 8 to 22 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butyryl, 1-pentynyl, 2-pentynyl, 3-methyl-1-butyryl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

As used herein, “heteroaryl” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkylthio” refers to an alkyl group as defined above attached through a sulfur bridge. An example of an alkylthio is methylthio, (i.e., —S—CH3).

“Alkoxy” refers to an alkyl group as defined above attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, and t-butoxy.

“Alkylamino” refers an alkyl group as defined above attached through an amino bridge. An example of an alkylamino is methylamino, (i.e., —NH—CH3).

“Alkanoyl” refers to an alkyl as defined above attached through a carbonyl bride (i.e., —(C═O)alkyl).

“Alkylsulfonyl” refers to an alkyl as defined above attached through a sulfonyl bridge (i.e., —S(═O)2alkyl) such as mesyl and the like, and “Arylsulfonyl” refers to an aryl attached through a sulfonyl bridge (i.e., —S(═O)2aryl).

“Alkylsulfinyl” refers to an alkyl as defined above attached through a sulfinyl bridge (i.e. —S(═O)alkyl).

The term “substituted” refers to a molecule wherein at least one hydrogen atom is replaced with a substituent. When substituted, one or more of the groups are “substituents.”

The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)2Ra, —OS(═O)2Ra and —S(═O)20Ra. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom to be unsubstituted.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In typical embodiments, the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Subject” refers any animal, preferably a human patient, livestock, rodent, monkey or domestic pet.

As used herein, the term “derivative” refers to a structurally similar compound that retains sufficient functional attributes of the identified analogue. The derivative may be structurally similar because it is lacking one or more atoms, substituted with one or more substituents, a salt, in different hydration/oxidation states, e.g., substituting a single or double bond, substituting a hydroxy group for a ketone, or because one or more atoms within the molecule are switched, such as, but not limited to, replacing an oxygen atom with a sulfur or nitrogen atom or replacing an amino group with a hydroxyl group or vice versa. Replacing a carbon with nitrogen in an aromatic ring is a contemplated derivative. The derivative may be a prodrug. Derivatives may be prepared by any variety of synthetic methods or appropriate adaptations presented in the chemical literature or as in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof

### (AcDP) Nanoparticles for Drug Delivery.

Self-assembled polymeric nanoparticles offer an alternative to the extremely high cost of antibody conjugates due to the practical difficulties of antibody-drug conjugation and manufacture without compromising efficacy. The selective delivery of anti-cancer agents into a tumor mass by nanoparticles can minimize toxicity to normal tissues and maximize bioavailability to cancer cells.

Nanocarriers for drug delivery should stay intact during the blood circulation, but once they reach the tumor sites the payload should be promptly released. The nanoparticles described herein act as tumor specific nanocarriers able to respond to the pH difference between blood (pH about 7.4) and acidic intracellular environment of cancer cell, such as the endosome and lysosome (pH=4-6.5).

Nanoparticles which reach the target site will preferably dissociate and allow the maytansinoid to interact with target cells, retaining cytotoxicity. In a preferable embodiment, pH-sensitive nanoparticles which do not reach the target site will be able to remain intact in blood for over 4 hours, or more preferably until such a time when the majority of the nanoparticle has been degraded at the target site or excreted from the body.

In addition to a change in stability of the nanoparticles at differing pH, the nanoparticle structure must also allow for an optimal biodistribution. In preferable embodiments, the nanoparticle will be comprised of dextran fragments with a molecular number of about 6000, modified to include polyethylene glycol chains with a molecular number of about 5000. Preferable embodiments will have hydroxyl groups modified to contain acetal groups, more preferably 1,1-dimethylacetal groups, as demonstrated in Formula I:

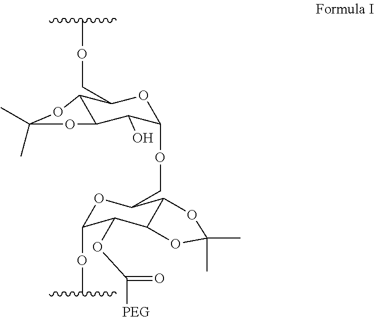

### Maytansinoids and Cytotoxic Compounds

In preferable embodiments, the nanoparticle will encapsulate maytansinoids of Formula I or salts thereof to exploit their extraordinary cytotoxicity to treat or inhibit the growth of cancerous or foreign proliferative growths.

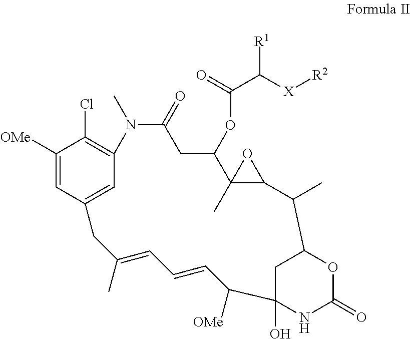

In preferable embodiments, R¹ and R² are each independently selected from hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R is optionally substituted with one or more of the same or different, J;

J is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl; and

X is any of oxygen, sulfur, or nitrogen; optionally substituted with one or more of the same or different J.

In preferable embodiments, the encapsulated maytansinoid will be maytansine, ansamitocin, mertansine or derivatives thereof. The nanoparticle may encapsulate a variety of other cytotoxic compounds, including but not limited to azathioprine, bleomycin, bortezomib, busulfan, capecitabine, carboplatin, carmustine, chlorambucil, cisplatin, cladribine, colaspase, cyclophosphamide, cytarabine, dacarbazine, dactinomycin, daunorubicin, docetaxel, doxorubicin, doxorubicin lisosomal, epirubicin, etoposide, fludarabine, fluorouracil, fotemustine, ganciclovir, gemcitabine, hydroxyurea, idarubicin, ifosamide, irinotecan, lomustine, melphalan, mercaptopurine, methotrexate, mitomycin, mitozantrone, oxaliplatin, paclitaxel, pemetrexed, procarbazine, raltitrexed, temozolomide, teniposide, thioguanine, thiotepa, topotecan, valganciclovir, vinblastine, vincristine, and vinorelbine.

### Formulations

Pharmaceutical compositions disclosed herein may include compounds in the form of pharmaceutically acceptable salts, as generally described below. Some preferred, but non-limiting examples of suitable pharmaceutically acceptable organic and/or inorganic acids are hydrochloric acid, hydrobromic acid, sulfuric acid, nitric acid, acetic acid and citric acid, as well as other pharmaceutically acceptable acids known per se (for which reference is made to the references referred to below).

When the compounds of the disclosure contain an acidic group as well as a basic group, the compounds of the disclosure may also form internal salts, and such compounds are within the scope of the disclosure. When a compound of the disclosure contains a hydrogen-donating heteroatom (e.g., NH), the disclosure also covers salts and/or isomers formed by the transfer of the hydrogen atom to a basic group or atom within the molecule.

Pharmaceutically acceptable salts of the compounds include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds can be administered by a variety of routes including the ocular, rectal, transdermal, subcutaneous, intravenous, intramuscular or intranasal routes, depending mainly on the specific preparation used. The compound will generally be administered in an “effective amount”, by which is meant any amount of a compound that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, more often between 0.1 and 500 mg, such as between 1 and 250 mg, for example about 5, 10, 20, 50, 100, 150, 200 or 250 mg, per kilogram body weight of the patient per day, which may be administered as a single daily dose, divided over one or more daily doses. The amount(s) to be administered, the route of administration and the further treatment regimen may be determined by the treating clinician, depending on factors such as the age, gender and general condition of the patient and the nature and severity of the disease/symptoms to be treated. Reference is made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences.

When administered by nasal aerosol or inhalation, the compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the disclosure or their physiologically tolerable salts in a pharmaceutically acceptable solvent, such as ethanol or water, or a mixture of such solvents. If required, the formulation may additionally contain other pharmaceutical auxiliaries such as surfactants, emulsifiers and stabilizers as well as a propellant.

For subcutaneous or intravenous administration, the compounds, if desired with the substances customary therefore such as solubilizers, emulsifiers or further auxiliaries are brought into solution, suspension, or emulsion. The compounds may also be lyophilized and the lyophilizates obtained used, for example, for the production of injection or infusion preparations. Suitable solvents are, for example, water, physiological saline solution or alcohols, e.g. ethanol, propanol, glycerol, sugar solutions such as glucose or mannitol solutions, or mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, the formulations may be prepared by mixing the compounds of formula I with a suitable non-irritating excipient, such as cocoa butter, synthetic glyceride esters or polyethylene glycols, which are solid at ordinary temperatures, but liquefy and/or dissolve in the rectal cavity to release the drug.

In certain embodiments, this disclosure contemplates a pharmaceutical composition comprising a nanoparticle, such as those described. Pharmaceutical compositions further comprising an anti-cancer agent are also considered. A pharmaceutical composition wherein the anti-cancer agent is selected from one or more groups of agents including of alkylating agents, antimetabolites, antibiotic anticancer agents, plant alkaloids, anthracenediones, natural products, hormones, hormones antagonists, miscellaneous agents, radiosensitizers, platinum coordination complexes, adrenocortical suppressants, immunosuppressive agent, functional therapeutic agents, gene therapeutic agents, antisense therapeutic agent, tyrosine kinase inhibitor, monoclonal antibody, immunotoxin, radioimmunoconjugate, cancer vaccine, interferon, interleukin, substituted ureas, taxanes and COX-2 inhibitors is considered.

In certain embodiments, this disclosure relates to a pharmaceutical composition wherein the anti-cancer agent is selected from one or more of chlormethine, chlorambucile, busulfan, thiotepa, chlorambucil, cyclophosphamide, estramustine, ifosfamide, meclilorethamine, melphalan, uramustine, carmπstine, lonuistine, streptozocin, dacarbazine, procarbazine, temozolainide, cisplatin, carboplatin, oxaliplatin, satraplatin, (SP-4-3)-(cis)-amminedichloro-[2-methylpyridine}-platinuni(II), methotrexate, permetrexed, raltitrexed, trimetrexate, cladribine, chlorodeoxyadenosine, clofarabine, fludarabine, mercaptopurine, pentostatin, thioguanine, azacitidine, capecltabine, cytarabine, edatrexate, floxuridine, 5-fluorouracil, genicitabine, troxacitabine, bleomycin, dactinomycin, adriamycin, actinomycin, mithramycin, mitomycin, mitoxantrone, porfiromycin, daunorubicin, doxorubicin, liposomal doxorubicin, epirubicin, idarubicin, valrubicin, phenesterine, tamoxifen, piposulfancamptothesin, L-asparaginase, PEG-L-asparaginase, paclitaxel, docetaxel, taxotere, vinblastine, vincristine, vindesine, vinorelbiiie, irinotecan, topotecan, amsacrine, etoposide, teniposide, fluoxymesterone, testolactone, bicalutamide, cyproterone, flutamide, nilutamide, aminoglutethimide, anastrozole, exemestane, formestane, letrozole, dexamethasone, prednisone, diethylstilbestrol, fulvestrant, raloxifene, tamoxifen, toremifme, buserelin, goserelin, leuprolide, triptorelin, medroxyprogesterone acetate, megestrol acetate, levothyroxine, liothyronine, altretamine, arsenic trioxide, gallium nitrate, hydroxyurea, levamisole, mitotane, octreotide, procarbazine, suramin, thalidomide, methoxsalen, sodium porfimer, bortezomib, erlotinib hydrochloride, gefitinib, imatinib mesylate, semaxanib, adapalene, bexarotene, trans-retinoic acid, 9-cis-retinoic acid, and N-(4-hydroxyplienyl)retinamide, alemtuzumab, bevacizumab, cetuximab, ibritumomab tiuxetan, rituximab, trastuzumab, gemtuzumab ozogamicin, tositumomab, interferon-α2a, interferon-α2b, aldesleukin, denileukin diftitox, and oprelvekin and derivatives thereof.

### Methods of Use

In certain embodiments, this disclosure also considers a method of treating cancer or inhibiting a proliferative growth comprising administering to a subject in need of such treatment an effective amount of a pharmaceutical composition disclosed herein. This disclosure also contemplates the method wherein the cancer is selected from of bladder cancer, breast cancer, colon cancer, endometrial cancer, leukemia, lung cancer, melanoma, non-Hodgkin lymphoma, pancreatic cancer, prostate cancer, rectal cancer, renal cell cancer, and thyroid cancer.

In certain embodiments, this disclosure relates to methods herein administering pharmaceutical products herein is done in combination with one or more anticancer agents.

The cancer treatments disclosed herein can be applied as a sole therapy or can involve, conventional surgery or radiotherapy, hormonal therapy, or chemotherapy. Such chemotherapy can include one or more of the following categories of anti-tumour agents:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulfan and nitrosoureas); antimetabolites (for example antifolates such as fluoropyrimidines like 5-fluorouracil and gemcitabine, tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin); and proteosome inhibitors (for example bortezomib [Velcade®]); and the agent anegrilide [Agrylin®]; and the agent alpha-interferon

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-Her2 antibody trastuzumab and the anti-epidermal growth factor receptor (EGFR) antibody, cetuximab), farnesyl transferase inhibitors, tyrosine kinase inhibitors and serine/threonine kinase inhibitors, for example inhibitors of the epidermal growth factor family for example EGFR family tyrosine kinase inhibitors such as: N-(3-chloro-4-fluorophenyl)-7-methoxy-6-(3-morpholinopropoxy)quinazolin-4-amine (gefitinib), N-(3-ethynylphenyl)-6,7-bis(2-methoxyethoxy)quinazolin-4-amine (erlotinib), and 6-acrylamido-N-(3-chloro-4-fluorophenyl)-7-(3-morpholinopropoxy)quinazolin-4-amine (CI 1033), for example inhibitors of the platelet-derived growth factor family and for example inhibitors of the hepatocyte growth factor family, for example inhibitors of phosphotidylinositol 3-kinase (PI3K) and for example inhibitors of mitogen activated protein kinase kinase (MEK1/2) and for example inhibitors of protein kinase B (PKB/Akt), for example inhibitors of Src tyrosine kinase family and/or Abelson (AbI) tyrosine kinase family such as dasatinib (BMS-354825) and imatinib mesylate (Gleevec™); and any agents that modify STAT signalling;

(v) antiangiogenic agents such as those which inhibit the effects of vascular endothelial growth factor, (for example the anti-vascular endothelial cell growth factor antibody bevacizumab [Avastin™]) and compounds that work by other mechanisms (for example linomide, inhibitors of integrin ocvβ3 function and angiostatin);

(vi) vascular damaging agents such as Combretastatin A4;

(vii) antisense therapies, for example those which are directed to the targets listed above, such as an anti-RAS antisense; and

(viii) immunotherapy approaches, including for example ex-vivo and in-vivo approaches to increase the immunogenicity of subject tumour cells, such as transfection with cytokines such as interleukin 2, interleukin 4 or granulocyte-macrophage colony stimulating factor, approaches to decrease T-cell energy, approaches using transfected immune cells such as cytokine-transfected dendritic cells, approaches using cytokine-transfected tumour cell lines and approaches using anti-idiotypic antibodies, and approaches using the immunomodulatory drugs thalidomide and lenalidomide [Revlimid®].

The combination therapy also contemplates use of the disclosed pharmaceutical compositions with radiation therapy or surgery, as an alternative, or a supplement, to a second therapeutic or chemotherapeutic agent.

A typical chronic lymphocytic leukemia (CLL) chemotherapeutic plan includes combination chemotherapy with chlorambucil or cyclophosphamide, plus a corticosteroid such as prednisone or prednisolone. The use of a corticosteroid has the additional benefit of suppressing some related autoimmune diseases, such as immunohemolytic anemia or immune-mediated thrombocytopenia. In resistant cases, single-agent treatments with nucleoside drugs such as fludarabine, pentostatin, or cladribine may be successful. Patients may consider allogeneic or autologous bone marrow transplantation. In certain embodiments, the disclosure contemplates combination treatments using compositions disclosed herein in combination with chloroambucil, cyclophosphamide, prednisone, prednisolone, fludarabine, pentostatin, and/or cladribine or combinations thereof.

Treatment of acute lymphoblastic leukemia typically includes chemotherapy to bring about bone marrow remission. Typical regiments include prednisone, vincristine, and an anthracycline drug, L-asparaginase or cyclophosphamide. Other options include prednisone, L-asparaginase, and vincristine. Consolidation therapy or intensification therapy to eliminate any remaining leukemia may include antimetabolite drugs such as methotrexate and 6-mercaptopurine (6-MP). In certain embodiments, the disclosure contemplates combination treatments using compositions disclosed herein in combination with COP, CHOP, R-CHOP, imatinib, alemtuzumab, vincristine, L-asparaginase or cyclophosphamide, methotrexate and/or 6-mercaptopurine (6-MP). COP refers to a chemotherapy regimen used in the treatment of lymphoma of cyclophosphamide, vincristine, and prednisone or prednisolone and optionally hydroxydaunorubicin (CHOP) and optionally rituximab (R-CHOP).

### Examples

### Synthesis of Dextran(6K)-PEG(5K)

Dextran (Mn=6000, 100 mg), PEG-COOH (Mn=5000, 100 mg), and 1,1′-carbonyldiimidazole (CDI) (3.9 mg, mole ratio of CDI:COOH=1.2:1) were dissolved in anhydrous DMSO. The reactions were allowed to stir at 80° C. overnight. The reaction mixtures were purified by dialyses against deionized water with Spectro/Por 6 bags (MWCO, 8K) for 3 days. The resulting solutions were lyophilized at −50° C. to offer the product as a white powder. GPC: Mn=11,300, PDI=1.6.

### Synthesis of Acetalated-Dextran-PEG(ADP)

Dextran(6K)-PEG(5K) (50 mg), 2-methoxypropene (1 ml), and pyridinium p-toluensulfonate (5 mg) were mixed in anhydrous DMSO. The reactions were allowed to stir at 20° C. for 3 h. The excess 2-methoxypropene was removed under reduced pressure. The reaction mixtures were further purified by dialyses against PBS (pH=7.4) with Spectro/Por 6 bags (MWCO, 8K) for 3 days. The resulting solutions were lyophilized at −50° C. to yield the product as a white powder.

### Synthesis of Propionic Anhydride Modified Dextran-PEG (PDP)

Dextran(6K)-PEG(5K) (50 mg), propionic anhydride (0.5 ml), dimethylaminopyridine (DMAP) (10 mg), and triethylamine (0.1 m) were added into anhydrous DMSO. The reaction was allowed to stir at RT for 5 h. The reaction mixtures were further purified by dialyses against deionized water with a Spectro/Por 6 bag (MWCO, 8K) for 3 days. The resulting solution was lyophilized at −50° C. to yield the product as a white powder.

### Preparation of ADP, PDP, and ADP-DM4Me Nanoparticles

ADP or PDP polymer were dissolved in DMSO, then added into PBS (pH=7.4) dropwise. The resulting mixtures were dialyzed against PBS (pH=7.4) for 24 h followed by lyophilization. ADP and PDP nanoparticles were obtained after re-suspending the dry powder in PBS. ADP-DM4Me nanoparticles were prepared as follows. Briefly, ADP (10 mg) and DM4Me (110 μg) were dissolved in DMSO (2 ml). DMSO was removed at 60° C. under reduced pressure (7 mmHg). After addition of PBS (pH=7.4, 15 ml), the mixture was sonicated with a Vibra-Cell (Sonics & Materials Inc.) for 10 min, and then centrifuged for 5 min at 2,000 rpm to remove the large particles. The supernatant was filtered with a 400 nm filter. The procedure for preparation of ADP-AP-3 and ADP-maytansine nanoparticles is same as ADP-DM4Me nanoparticles.

### In Vivo Antitumor Activities of DM4Me and ADP-DM4Me.

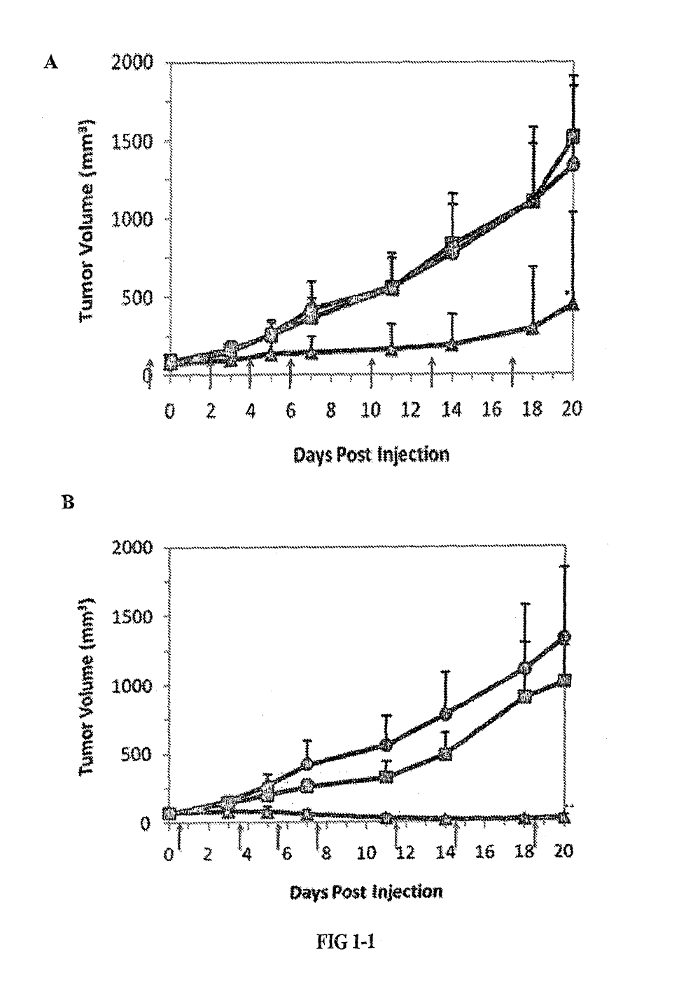

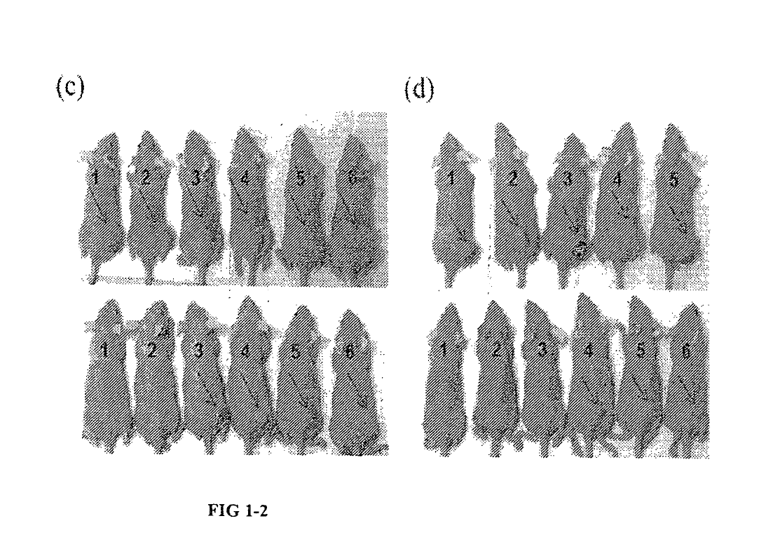

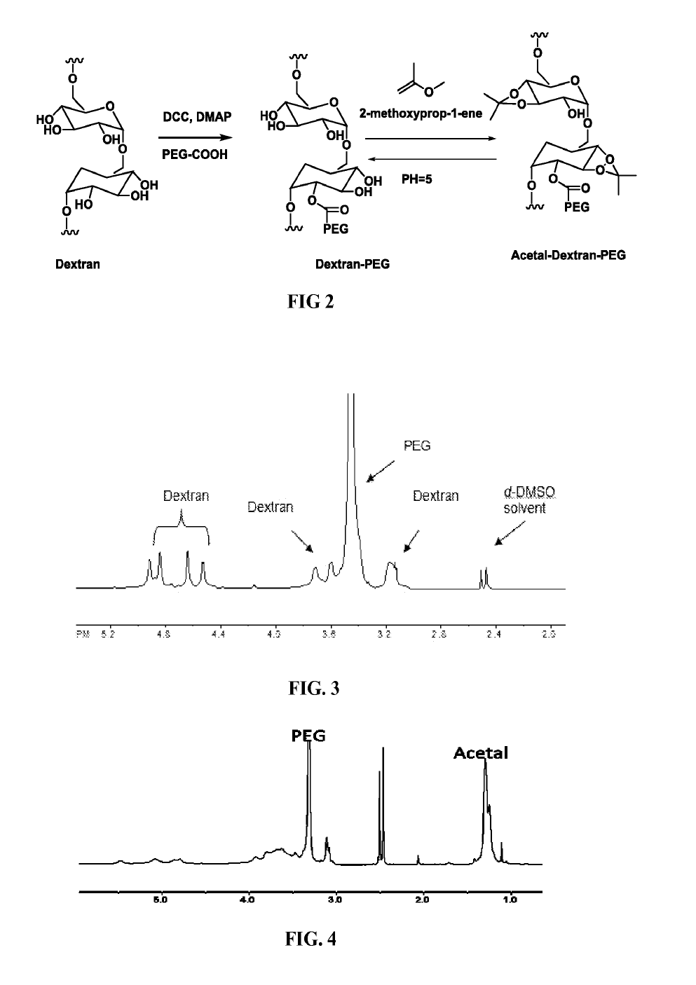

Mice treated with DM4Me showed no response when treated with 0.5 mg/kg (FIG. 1a, blue line). Two of the five mice treated (1 mouse accidentally died during the injection) showed a partial response when the dosage was increased to 0.75 mg/kg (slower tumor growth rate than control; FIG. 1b, blue line). Of the six mice treated with 0.5 mg/kg ADP-DM4Me, 2 showed completely healing, 3 of 6 mice showed partially response (slower tumor growth rate compared with free drug treated group mice) and 1 mouse showed no response (FIG. 1a, red line). At a dosage of 0.75 mg/kg ADP-DM4Me, 3 of 6 mice showed completely healing and the rest of mice showed tumor shrinkage during the treatments (none of those mice's tumor size increased; FIG. 1a, red line). Photographs of each group are shown in FIG. 1c (0.5 mg/kg doses of DM4Me and ADP-DM4Me, top and bottom respectively), and FIG. 1d (0.75 mg/kg doses of DM4Me and ADP-DM4Me, top and bottom respectively).

1. A particle comprising a maytansinoid and an acetalated polysaccharide-polyethylene glycol conjugate.

2. The particle of claim 1 wherein the acetalated polysaccharide-polyethylene glycol comprises monomers of Formula I:

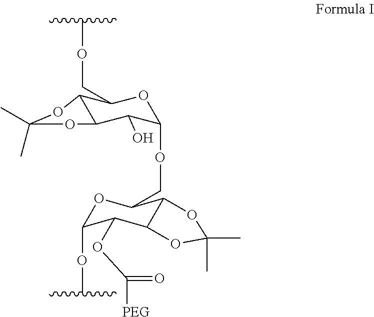

3. The particle of claim 1 with a size of around 40-400 nanometers.

4. The particle of claim 1 wherein the acetalated polysaccharide-polyethylene glycol comprises polyethylene glycol fragments with a molecular number of about 4-6 kilodaltons.

5. The nanoparticle of claim 1 wherein the acetalated polysaccharide-polyethylene glycol comprises of a dextran polymer with a molecular number of about 5-7 kilodaltons.

6. The nanoparticle of claim 1 wherein the acetalated polysaccharide-polyethylene glycol is stable at physiological pH and unstable in mildly acidic conditions.

7. The nanoparticle of claim 1 wherein the maytansinoid is selected from maytansine, ansamitocin, or mertansine.

8. The nanoparticle of claim 1 wherein the maytansinoid is a compound of Formula II: 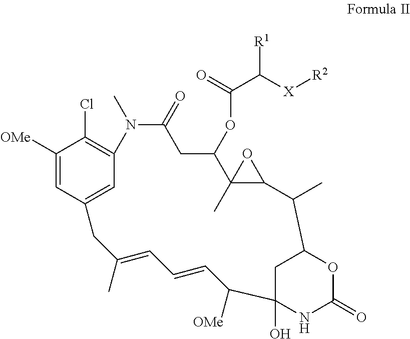
or salts thereof; wherein
R1 and R2 are each independently selected from hydrogen, alkyl, halogen, nitro, cyano, hydroxy, amino, mercapto, formyl, carboxy, alkanoyl, carbamoyl, alkoxy, alkylthio, alkylamino, (alkyl)2amino, alkylsulfinyl, alkyl sulfonyl, aryl sulfonyl, carbocyclyl, aryl, heterocyclyl, polypeptide, or antibody, wherein R1 and R2 are optionally substituted with one or more of the same or different, J; and wherein
J is alkyl, halogen, nitro, cyano, hydroxy, amino, mercapto, formyl, carboxy, alkanoyl, carbamoyl, alkoxy, alkylthio, alkylamino, (alkyl)2amino, alkylsulfinyl, alkyl sulfonyl, arylsulfonyl, carbocyclyl, aryl, or heterocyclyl; and wherein
X is any of oxygen, sulfur, or nitrogen; optionally substituted with one or more of the same or different J.

9. The nanoparticle of claim 8 wherein X is nitrogen.

10. The nanoparticle of claim 8 wherein X is nitrogen substituted with a methyl group.

11. The nanoparticle of claim 8 wherein R2 is carboxymethyl.

12. The nanoparticle of claim 8 wherein R1 is methyl.

13. A pharmaceutical composition comprising a nanoparticle of claim 1.

14. The pharmaceutical composition of claim 13 further comprising an anti-cancer agent.

15. A method of treating cancer or inhibiting a proliferative growth comprising administering to a subject in need of such treatment an effective amount of a pharmaceutical composition as described in claim 13.

16. The method of claim 15 wherein the cancer is selected from of bladder cancer, breast cancer, colon cancer, endometrial cancer, leukemia, lung cancer, melanoma, non-Hodgkin lymphoma, pancreatic cancer, prostate cancer, rectal cancer, renal cell cancer, and thyroid cancer.
